SEQUENCE LISTING (2 sequences)

SEQ ID NO: 1 - protein, 7 aa, Artificial Sequence
  Synthetic polypeptide
PLGLXAR

SEQ ID NO: 2 - protein, 19 aa, Artificial Sequence
  Synthetic polypeptide
XKKKKVLPIQLNAATDKGG

(19) United States
(12) Patent Application Publication
(10) Pub. No.: US 2012/0135959 A1
(43) Pub. Date: May 31, 2012

(54) Metalloprotein inhibitors containing nitrogen based ligands

(76) Inventors: Seth M. Cohen, San Marcos, CA (US); Faith E. Jacobsen, Bloomington, IN (US); Jana A. Lewis, Nashville, TN (US)

(21) Appl. No.: 11/888,237

(22) Filed: Jul. 30, 2007

Related U.S. Application Data
(60) Provisional application No. 60/834,634, filed on Jul. 31, 2006.

(51) Int. Cl. A61K 31/69; C12N 9/99; A61P 35/00; A61P 29/00; A61P 37/02; A61P 31/10; A61P 33/00; A61P 1/16; A61P 9/10; A61P 37/08; A61P 25/14; A61P 27/02; A61P 33/02; A61P 7/06; C07F 5/02

(52) U.S. Cl. 514/64; 548/110; 435/184

(57) ABSTRACT

Provided herein metalloproteinase inhibitors containing nitrogen or mixed nitrogen/oxygen donating zinc binding groups. Also provided are pharmaceutical compositions containing the compounds and methods of treating preventing or ameliorating the diseases associated with metalloproteinase activity using the compounds and compositions.

### RELATED APPLICATIONS

Priority is claimed herein to U.S. Provisional Patent Application No. 60/834,634, filed Jul. 31, 2006, entitled “METALLOPROTEIN INHIBITORS CONTAINING NITROGEN BASED LIGANDS.” The disclosure of the above-referenced provisional application is incorporated by reference herein in its entirety.

### FIELD

Provided herein are metalloprotein inhibitors containing amine ligands, compositions thereof and methods of use of the compounds and compositions.

### BACKGROUND

MMPs are a class of zinc(II)-containing hydrolytic enzymes involved in the breakdown of the extracellular matrix and the basement-membrane including components such as aggrecan, collagen, elastin, fibronectin, gelatin, and laminin. The ability of MMPs to degrade components of the extracellular matrix is essential to tasks such as cell growth, cell division, bone growth, wound healing, embryogenesis, and angiogenesis. Disruption of the regulation of MMP activity is correlated to disease states including but not limited to cardiovascular disease, stroke, arthritis, and tumor metastasis. Many factors must be considered in designing an effective and selective drug. In the case of matrix metal liproteinase inhibitors (MMPi), the drug typically has two parts, a peptidomimetic backbone and a zinc-binding group (ZBG). The backbone serves as a substrate analogue, allowing the inhibitor to fit in the active-site cleft of the enzyme. The ZBG binds to the catalytic zinc(II) ion, thereby rendering the MMP inactive. The vast majority of MMPi investigations have focused on improving the backbone interactions of MMPi while opting to use a well-known ZBG, namely a hydroxamic acid moiety, that has been in regular use for more than 20 years. Hydroxamates are indiscriminate metal chelators, known to bind a variety of metal ions, including Fe³⁺. Although extensive efforts have been made to improve MMPi by manipulating the substrate-like backbone of the drug, significantly fewer efforts have concentrated on improving the ZBG. Thus, there is a need for the identification of more potent and selective ZBGs to take MMPi toward a more productive second generation of development.

Histone deacetylases (HDACs) and the silent information regulator-like family of NAD-dependent deacetylases are important in transcriptional regulation. Acetylation neutralizes the lysine charge, and DNA unwinds, thus allowing active gene expression to occur. Deacetylation leads to the packing of nucleosomes as chromatins and thus gene repression. HDACs deacetylate using an activated water in

the active site where two glutamic acid residues and a histidine residue are coordinated to an active site metal ion with a histidine-aspartate charge-relay system. Anomalous HDAC activity has been associated with cancer, and HDAC inhibitors have been proposed as cancer treatments. HDAC inhibitors generally have a form of a ZBG to bind the catalytic zinc ion, a linker to interact with the narrow channel leading down to the active site, and a surface recognition or capping group that will interact with the surface of the protein. Some of the HDACi in the literature include short chain fatty acids like valproic acid, hydroxamates like trichostatin A, cyclic hydroxamic-acid-containing peptide compounds, epoxides, and benzamidines.

Anthrax spores are taken up by alveolar macrophages and germinate in the lymphnodes where the spores create toxins to inhibit immune responses. Anthrax is often asymptomatic until it reaches the blood, and then it is often fatal and non-responsive to traditional antibiotics. In order for an anthrax infection to be toxic, the protective antigen (PA) must form a heptamer that will mediate entry of up to three molecules of edema factor (EF) and lethal factor (LF) per heptamer into cells. Anthrax lethal factor is one of three proteins involved in anthrax pathogenesis and lethality. Inactivation of the LF gene in B. anthracis leads to a thousand-fold or greater reduction of virulence, which suggests that anthrax pathology is largely determined by LF. The active site of anthrax lethal factor consists of two histidine residues and a glutamic acid residue bound to a zinc(II) ion. Again, many known LF inhibitors contain a hydroxamate as a ZBG, and some proposed inhibitors are based on animoglycosides, small peptides attached to a ZBG, or were identified from the NCI Diversity Set.

Therefore, there is a continuing need for metalloproteinase inhibitors (MPI), such as matrix metalloproteinase inhibitors (MMPi), histone deacetylase inhibitors (HDACi), or anthrax lethal factor inhibitors (LFi) with better zinc binding groups.

### SUMMARY

Provided herein are metalloprotein inhibitors containing nitrogen and mixed nitrogen/oxygen donor-atom in the zinc binding groups. In certain embodiments, the compounds are inhibitors of metalloproteinase, histone deacetylases or anthrax lethal factor. In certain embodiments, the compounds provided herein have improved affinity and selectivity for the active site Zn²⁺ ion in the metalloprotein inhibitor as compared with the metalloprotein inhibitor compounds that contain other zinc binding groups (ZBGs) known in the art, such as hydroxamate ZBGs, carboxylic acid ZBGs, thiol ZBGs or phosphorus-based ZBGs. In certain embodiments, the zinc binding groups for use in the compounds provided herein are chosen for their known binding affinity and preference for late transition metals (e.g., Cu²⁺, Zn²⁺) over earlier transition metal and group I/II metal ions. Belal, et al. J. Chem. Eng. Data 1997, 42, 1075-1077; Arishima, et al. Nippon Kagaku Kaishi 1973, 6, 1119-1121 and Kawabata, et al., J. Am. Chem. Soc. 2005, 127, 818-819.

In certain embodiments, the compounds provided herein have Formula I:

(R)ₙ-L-ZBG,

wherein R is a chemical backbone for generating noncovalent interactions within the metalloprotein active site pocket;

n is 1-4;

L is a linker and ZBG is a zinc binding group containing nitrogen or mixed nitrogen/oxygen donor-atom.

In certain embodiments, the ligands for use as the ZBGs in the metalloprotein inhibitors provided herein are

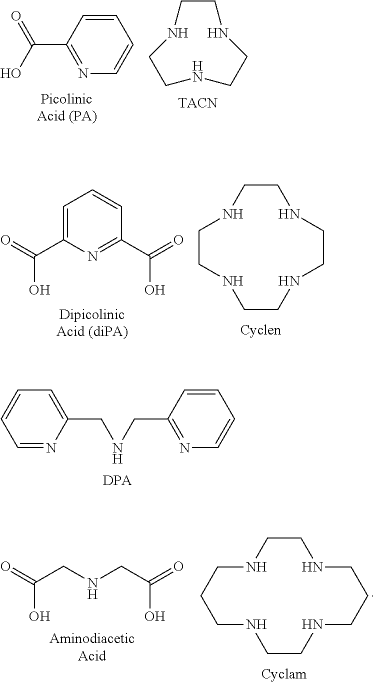

In certain embodiments, the chemical backbone for generating noncovalent interactions within the metalloprotein active site pocket contains a peptide, peptidomimetic moiety or other suitable moiety for such interactions. Several chemical backbones suitable for such interactions are known in the art, for example see, Rao G., Curr Pharm Des. 2005; 11(3):295-322; Whittaker, et. al. Chem. Rev. 1999, 99, 2735-2776; International Application Publication No. WO 2006/028523 and U.S. Application Publication No. 2005/0267102.

Also provided are pharmaceutically-acceptable derivatives, including salts, esters, enol ethers, enol esters, solvates, and hydrates of the compounds described herein. Further provided are pharmaceutical compositions containing a compound provided herein and a pharmaceutically acceptable carrier. In one embodiment, the pharmaceutical compositions are formulated for single dosage administration.

Methods of treatment of diseases associated with metalloprotein, including, but not limited to metalloproteinases, such as matrix metalloproteinases, (MMPs), histone deacetylases (HDAC), and anthrax lethal factor (LF). The diseases include, but not limited to, proliferative diseases, autoimmune diseases, infectious diseases and inflammatory diseases. For example, diseases include e, but are not limited to, rheumatoid arthritis, lupus, multiple sclerosis, psoriasis, diabetic retinopathies, other ocular disorders, including recurrence of pterygii, scarring excimer laser surgery and glaucoma filtering surgery, various disorders of the anterior eye, cardiovascular disorders, restenosis, chronic inflammatory diseases, wounds, circulatory disorders, crest syndromes, bacterial infections, viral diseases, including AIDS, dermatological disorders, and cancer, including solid neoplasms and vascular tumors, including, but are not limited to, lung, colon, esophageal, breast, ovarian and prostate cancers.

In one embodiment, diseases associated with histone deacetylases, include, but are not limited to, cancer; psoriasis; fibroproliferative disorders; smooth muscle cell proliferation disorders; inflammatory diseases and conditions treatable by immune modulation; neurodegenerative disorders; diseases involving angiogenesis; fungal and parasitic infections; haematopoietic disorders; liver fibrosis; arteriosclerosis; restenosis; rheumatoid arthritis; autoimmune diabetes; lupus; allergies; Huntington's disease; retinal diseases, such as diabetic retinopathy, age-related macular degeneration, interstitial keratitis or rubeotic glaucoma; protozoal infections; anaemia; sickle cell anaemia; thalassemia and protozoal infections, such as malaria, toxoplasmosis or coccidiosis.

Articles of manufacture are provided containing packaging material, a compound or composition provided herein which is useful as a metalloprotein inhibitor, and a label that indicates that the compound or composition is useful a metalloprotein inhibitor.

### BRIEF DESCRIPTION OF FIGURES

FIG. 1 illustrates Chemical (left) and structural (right, 50% probability ellipsoids) diagram of [(Tp^{Ph,Me})Zn(1)] showing bidentate chelation of the ligand to the zinc(II) ion. Hydrogen atoms and solvent have been omitted for clarity.

FIG. 2 shows best fit orientation of ZBG 1 superpositioned into the MMP active site (left) and the LF active site (right). T=25° C.

FIG. 3 illustrates a comparison of maltol, 1 (picolinic acid), and 5 (TACN) superpositioned into the MMP-3 (1G4K) active site.

FIG. 4 provides two views of ZBG 5 (TACN) in the active site of MMP-3, showing that this macrocyclic ZBG is not too large to access the active site metal ion in this protein. For the superposition of 5 the structure of the homoleptic complex [Zn(L)₂]²⁺ was utilized (Chaudhuri et al. Inorg. Chem. 1992, 31, 1457-1462).

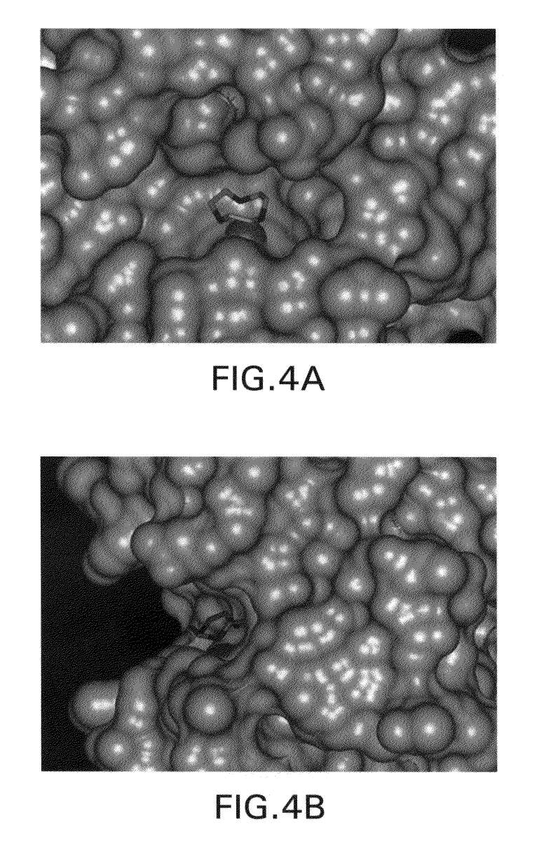

### DETAILED DESCRIPTION

### A. Definitions

Unless defined otherwise, all technical and scientific terms used herein have the same meaning as is commonly understood by one of ordinary skill in the art to which the claimed subject matter belongs. All patents, applications, published applications and other publications are incorporated by reference in their entirety. In the event that there are a plurality of definitions for a term herein, those in this section prevail unless stated otherwise.

As used herein, chemical backbone refers to a moiety that is capable of generating noncovalent interactions within the metalloprotein active site pocket. Several such moieties are known to one of skill in the art. Exemplary moieties are set forth in comprehensive reviews by Rao G., Curr Pharm Des. 2005; 11(3):295-322; and Whittaker, et. al. Chem. Rev. 1999, 99, 2735-2776. Exemplary of such chemical backbone moieties are described in the instant specification. Those of skill in the art will appreciate that a wide array of backbone structures that can be used in the compounds provided herein. For example, backbone moieties of use herein can be peptidic or peptidomimetic groups. Some exemplary scaffold moieties are described elsewhere herein and in International Application Publication No. WO 2006/028523, which is incorporated by reference in its entirety.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, and cycloalkyl. Pharmaceutically acceptable enol esters include, but are not limited to, derivatives of formula C═C(OC(O)R) where R is hydrogen, alkyl, alkenyl, alkynyl, or cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the claimed subject matter encompasses any racemic, optically active, polymorphic, or steroisomeric form, or mixtures thereof, of a compound provided herein, which possesses the useful properties described herein, it being well known in the art how to prepare optically active forms and how to determine antiproliferative activity using the standard tests described herein, or using other similar tests which are will known in the art.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-penytyl and isohexyl. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or Spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q1.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “haloalkoxy” refers to RO in which R is a haloalkyl group.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH— CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “substituted alkylene,” “substituted alkenylene,” “substituted alkynylene,” “substituted cycloalkylene,” “substituted cycloalkenylene,” and “substituted cycloalkynylene” refer to alkylene, alkenylene, alkynylene, cycloalkylene, cycloalkenylene and cycloalkynylene groups, respectively, that are substituted with one or more substituents, in certain embodiments one to three or four substituents, where the substituents are as defined herein.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 5 or 6 carbons.

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three of four substituents, where the substituents are as defined herein. As used herein, the term “carbamyl” refers to a functional group in which a nitrogen atom is directly bonded to a carbonyl, such as NH₂CO—.

Where the number of any given substituent is not specified (e.g., “haloalkyl”), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein “subject” is an animal, such as a mammal, including human, such as a patient.

As used herein, the term “parenteral” includes subcutaneous, intravenous, intrathecal, intra-arterial, intramuscular or intravitreal injection, or infusion techniques.

The term “topically” encompasses administration rectally and by inhalation spray, as well as the more common routes of the skin and mucous membranes of the mouth and nose and in toothpaste.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944). Certain of the abbreviations used herein are as follow:

ZBG=zinc binding group

ZBG 1 or 1=picolinic acid

ZBG 2 or 2=triazacyclononane (TACN)

ZBG 3 or 3=dipicolenic acid (DIPA)

ZBG 4 or 4=cyclen

ZBG 5 or 5=di(2-picolyl)amine (DPA)

ZBG 6 or 6=aminodiacetic acid

ZBG 7 or 7=cyclam

AHA=acetohydroxamic acid

### B. Compounds

Provided herein are metalloprotein inhibitors containing nitrogen or mixed nitrogen/oxygen donor-atom chelators, including, but not limited to, pyridine- and aza-macrocycle-based ligands as zinc binding groups (ZBGs). In certain embodiments, the compounds are inhibitors of metalloproteinase, histone deacetylases or anthrax lethal factor. In certain embodiments, the compounds provided herein have improved affinity and selectivity for the active site Zn²⁺ ion in the metalloprotein compared with the metalloprotein inhibitor compounds that contain other ZBGs known in the art, such as hydroxamate ZBGs, carboxylic acid ZBGs, thiol ZBGs or phosphorus-based ZBGs.

In certain embodiments, the zinc binding groups for use in the compounds provided herein are chosen for their known binding affinity and preference for late transition metals (e.g., Cu²⁺, Zn²⁺) over earlier transition metal and group I/II metal ions. Methods to determine the binding affinity of ligands for use in the compounds provided herein are known in the art.

In certain embodiments, the compounds provided herein have Formula I:

(R)ₙ-L-ZBG,

wherein R is a chemical backbone for generating noncovalent interactions within the metalloprotein active site pocket;

L is a linker that connects the chemical backbone with the zinc binding group;

n is 1-4; and

ZBG is a zinc binding group containing nitrogen or mixed nitrogen/oxygen donor-atom chelators.

In certain embodiments, the compounds provided herein have Formula I:

R-L-ZBG,

wherein the variables are as defined elsewhere herein.

In certain embodiments, the ligands for use as the ZBGs in the metalloproteinase inhibitors are nitrogen donor atom. In certain embodiments, the ligands for use as the ZBGs in the metalloproteinase inhibitors provided herein are mixed nitrogen/oxygen donor-atoms. In certain embodiments, the ZBGs are

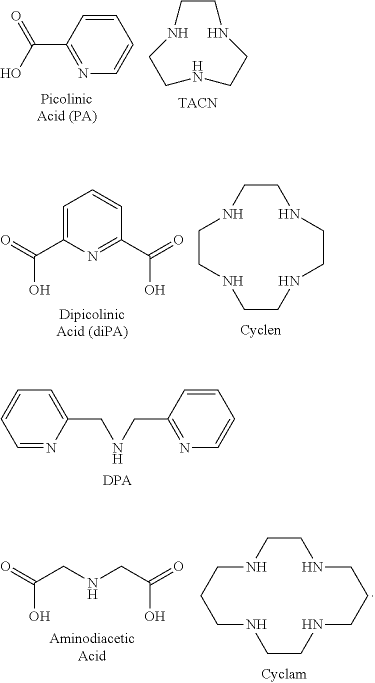

In certain embodiment, R is a chemical backbone for generating noncovalent interactions within the metalloproteinase active site pocket. In certain embodiments, the chemical backbone for generating noncovalent interactions within the metalloprotein active site pocket contains a peptide, peptidomimetic moiety or other suitable moiety for such interactions. Several suitable backbones for such interactions are known in the art, for example see, Rao G., Curr Pharm Des. 2005; 11(3):295-322; Whittaker, et. al. Chem. Rev. 1999, 99, 2735-2776; European Patent Application No. 126,974; International Application Publication No. WO 2006/028523 and U.S. Application Publication No. 2005/0267102.

The structure of the organic backbone molecule(s) and other organic substituents is selected such that it do not interfere with and, in certain embodiments, enhance the ability of the metalloprotein inhibitor to direct the ZBG toward one or more complexed metal ions, such as Zn(II) atoms of the MMP. For example, R can be any of the organic radicals derived from the structures shown on Scheme 1 of the Whittaker, et. al. Chem. Rev. 1999, 99, 2735-2776, after removal of the C(O)NH(OH) group. The organic backbone molecule and/or substituent(s) can be a naturally-occurring peptide, a synthetic peptide or a peptide analog (peptidomimetic). Such groups may contain one or more amido moieties (—C(O)NH—), which can be or contain, peptidyl bonds.

Exemplary chemical backbones suitable for use in the compounds provided herein are illustrated below. In the compounds described below, the ZBGs are for example, hydroxamate ZBGs, carboxylate ZBGs, thiol ZBGs and phosphorus-based ZBGs and others. In certain embodiments, the compounds of Formula I provided herein are obtained by replacing the ZBGs from the compounds described below by the nitrogen or mixed nitrogen/oxygen donor ZBGs. The ZBG provided herein can be linked to the chemical backbone via a linker.

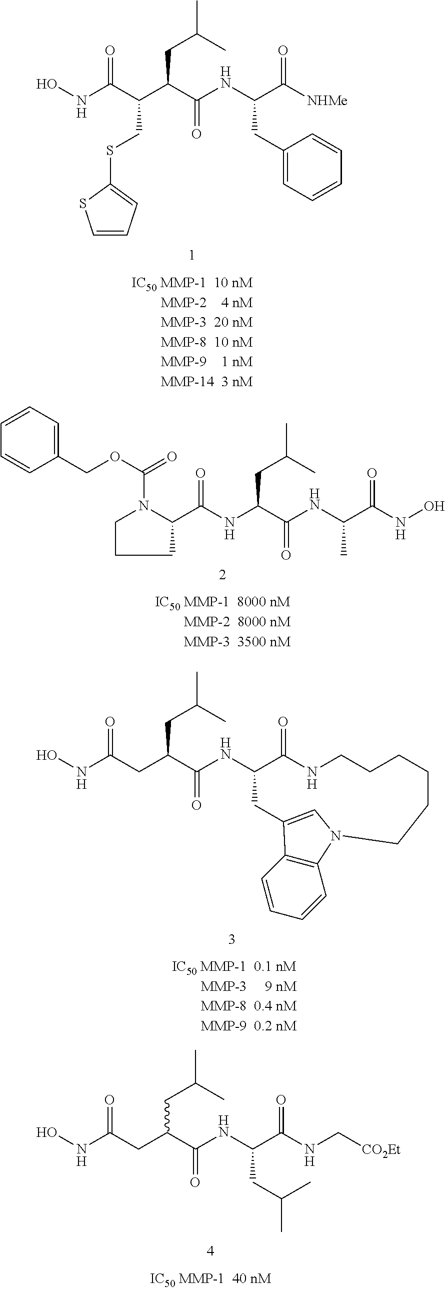

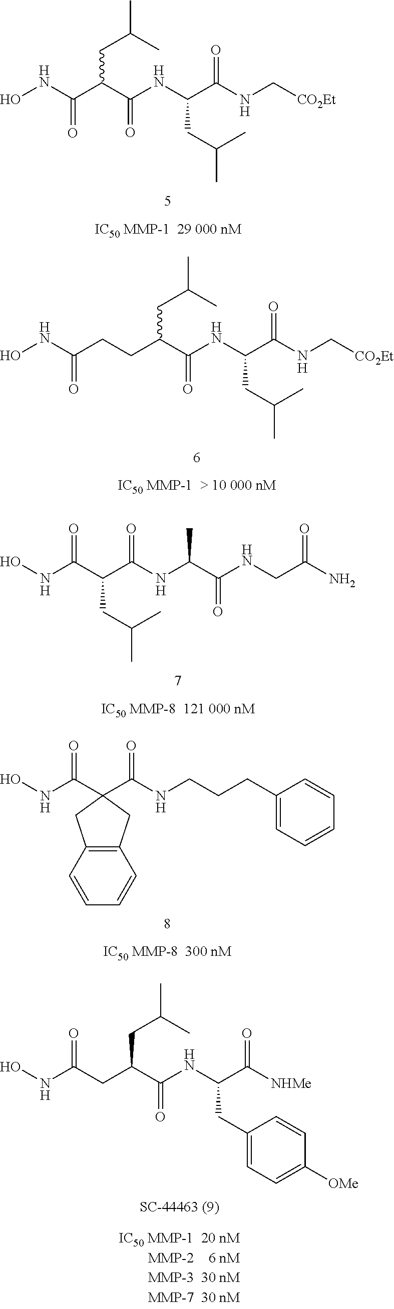

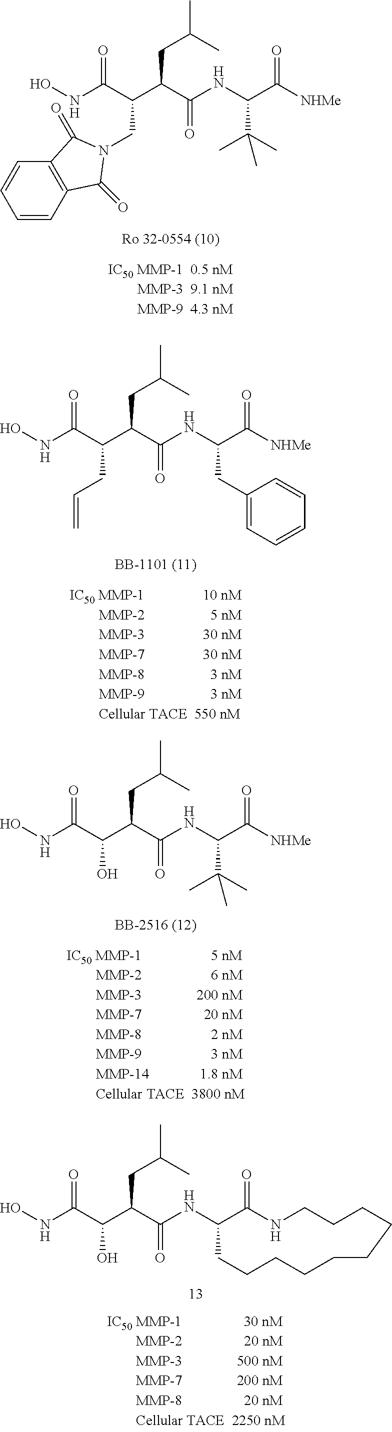

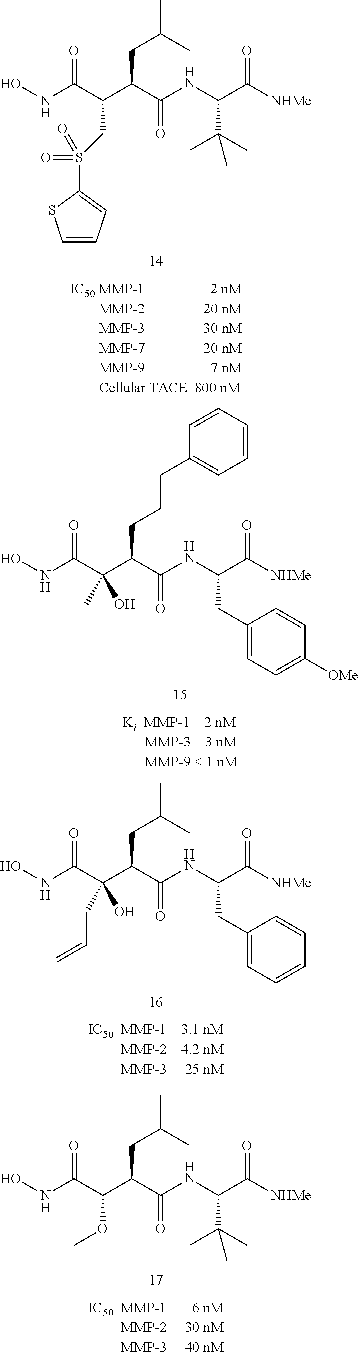

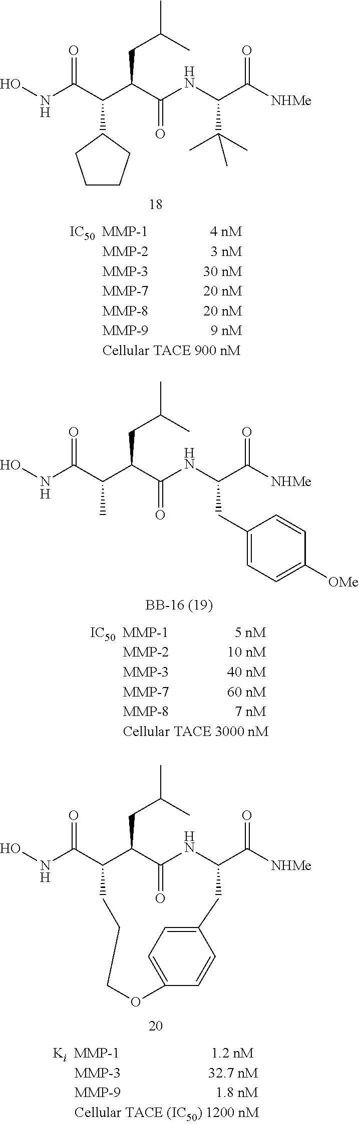

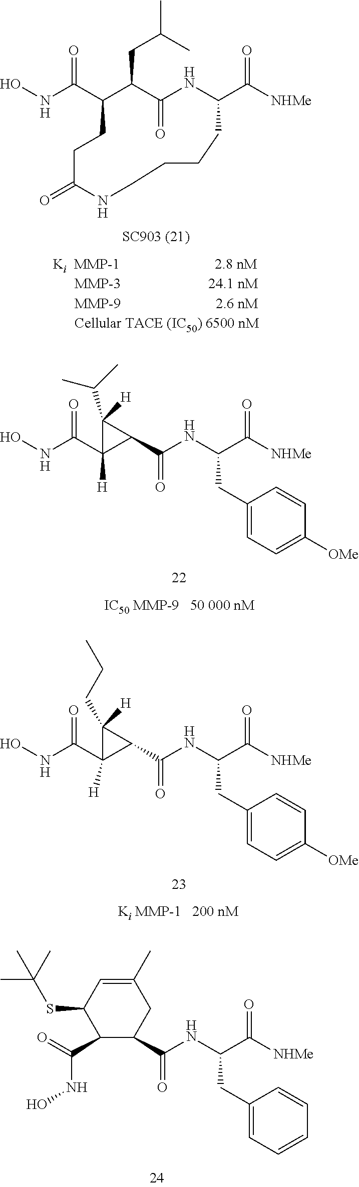

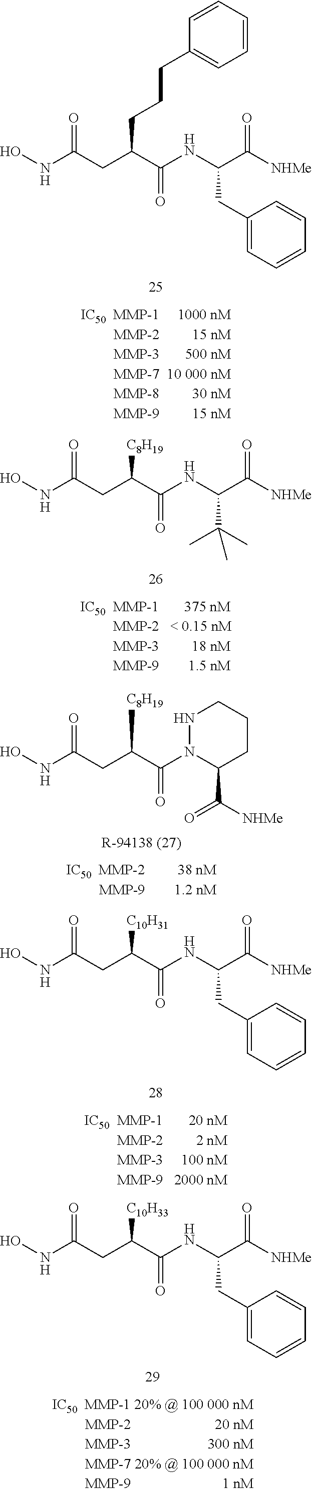

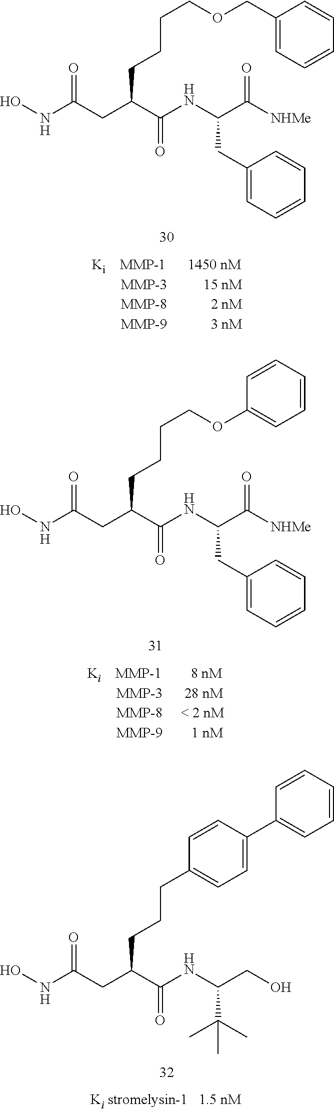

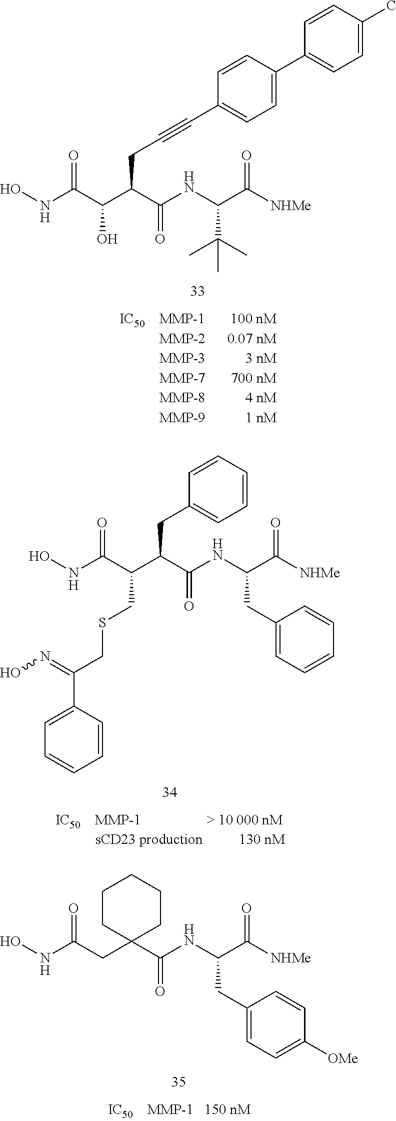

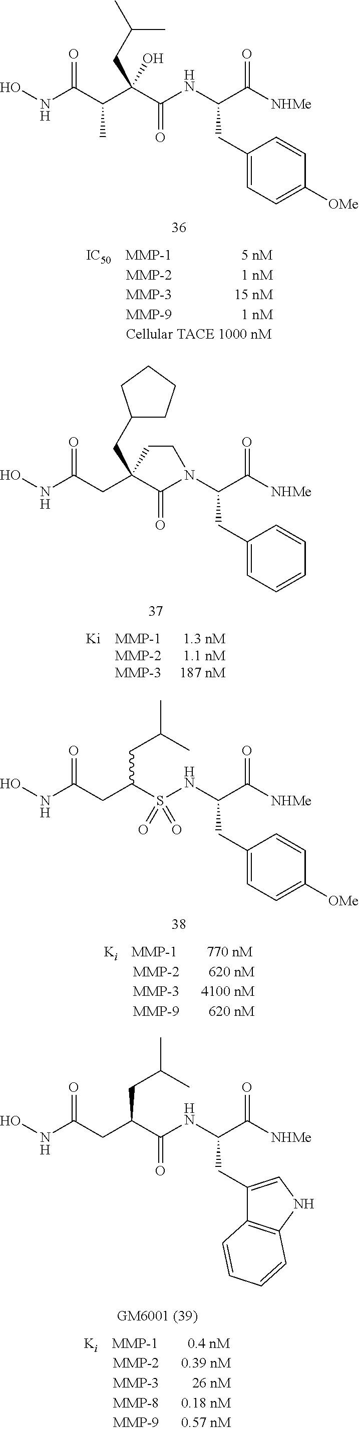

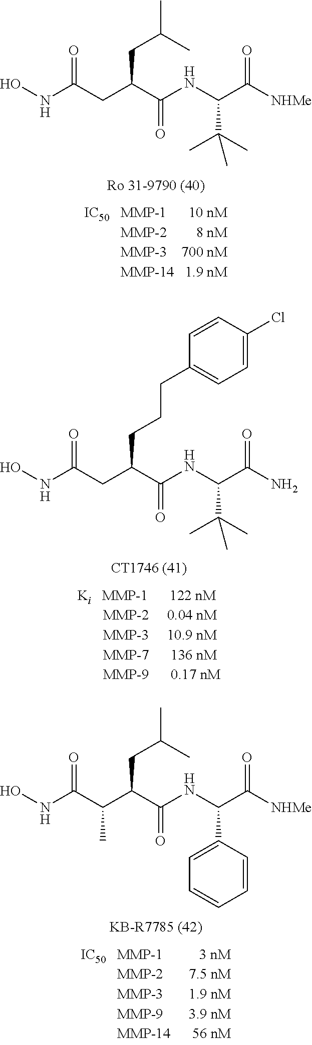

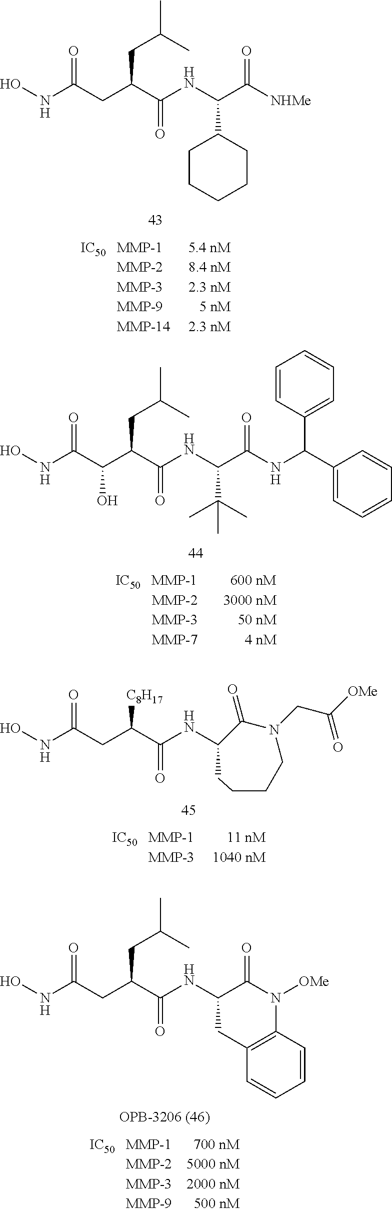

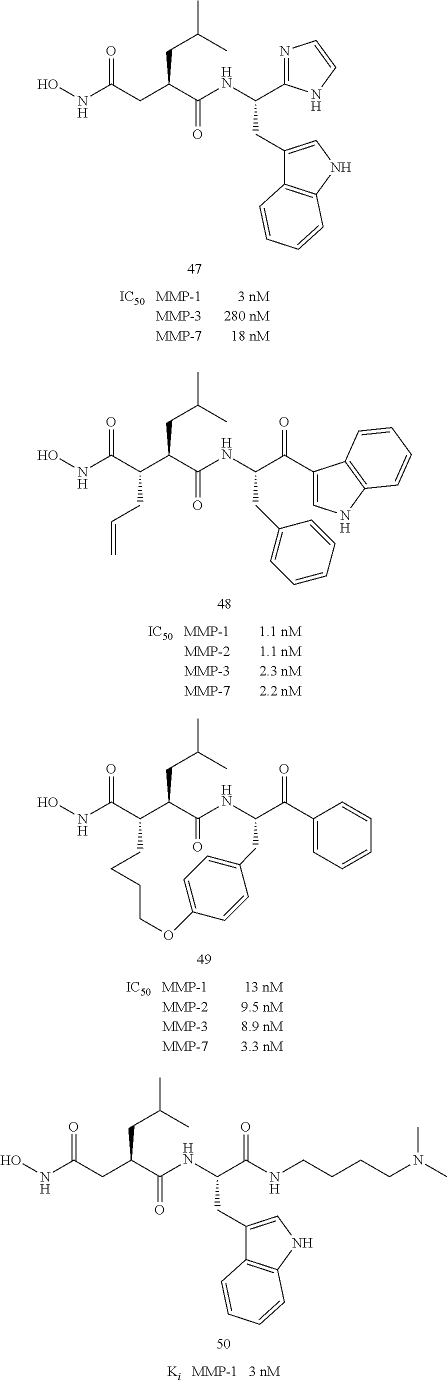

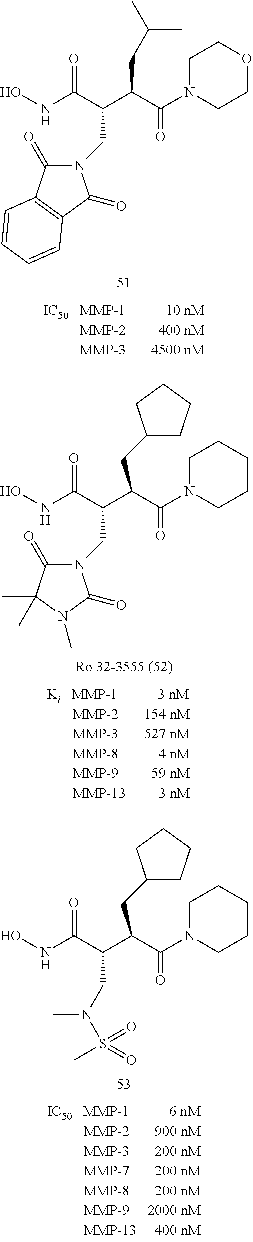

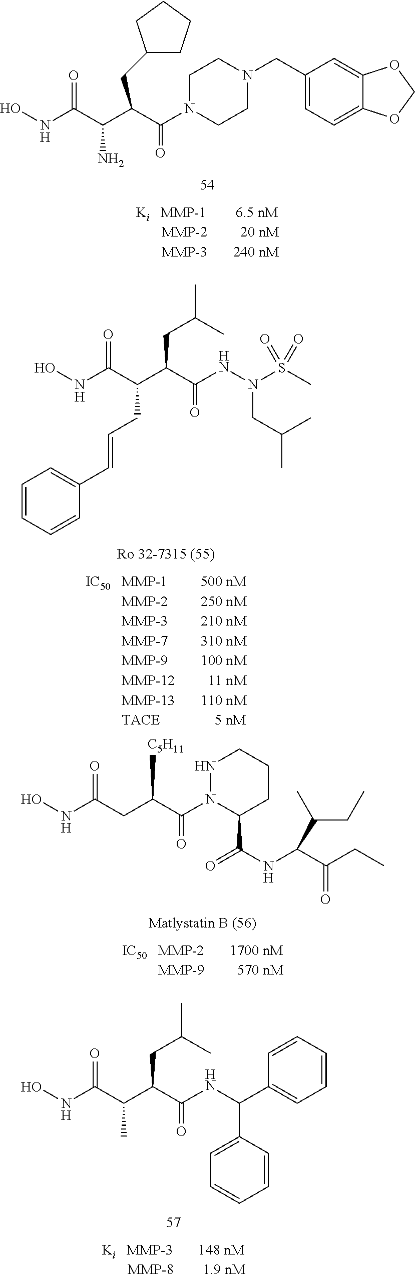

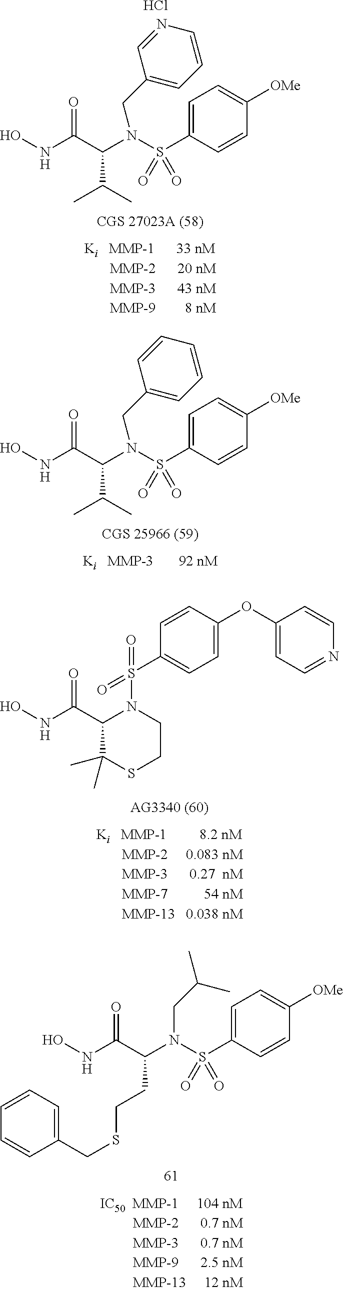

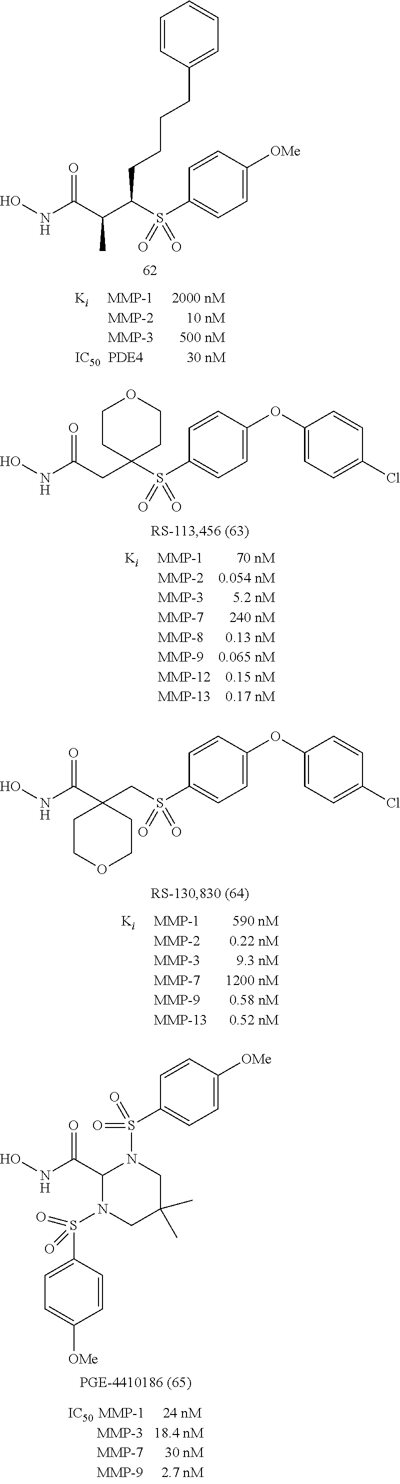

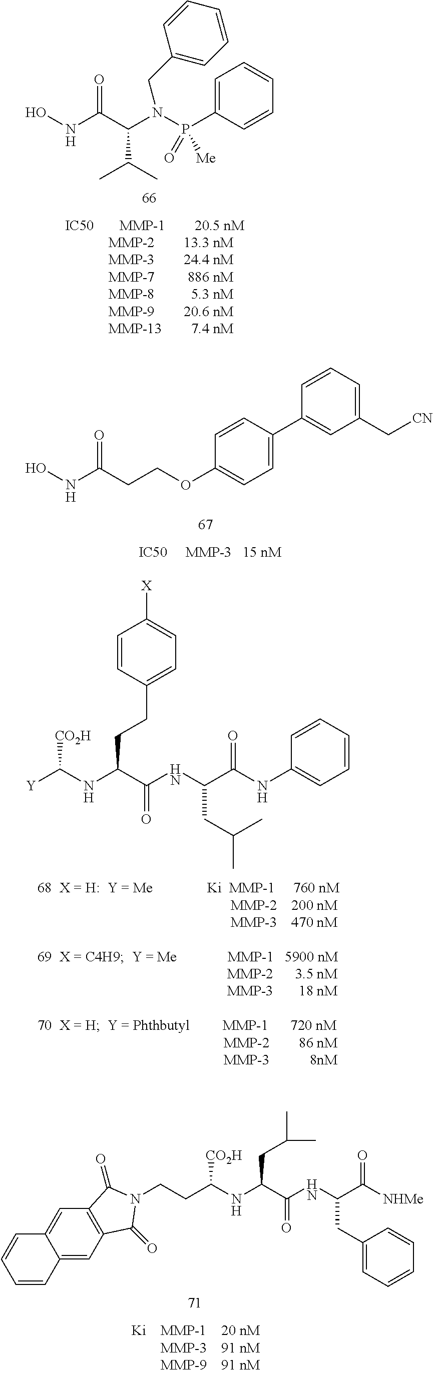

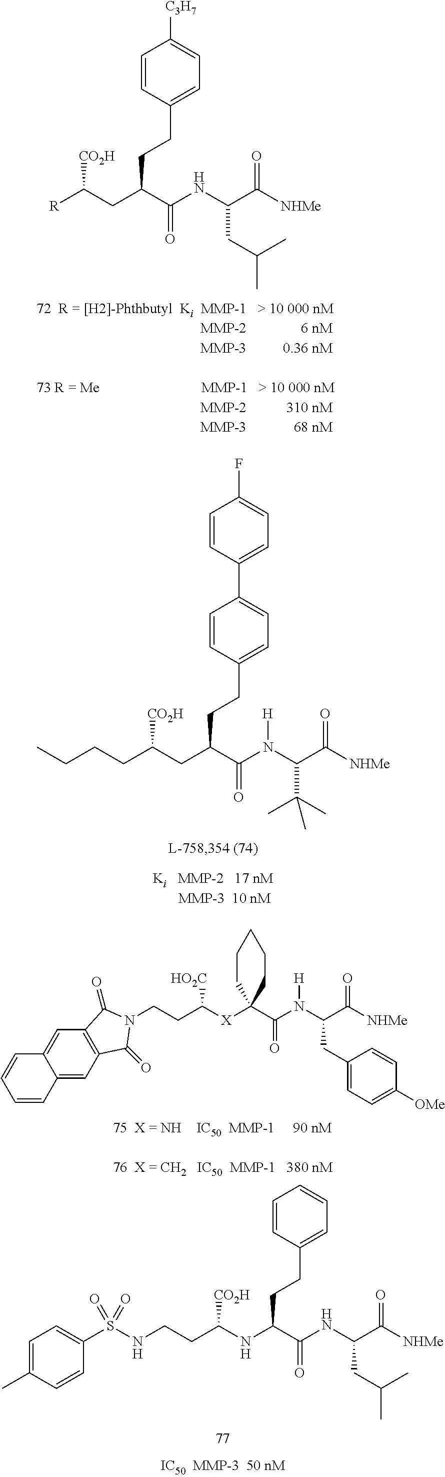

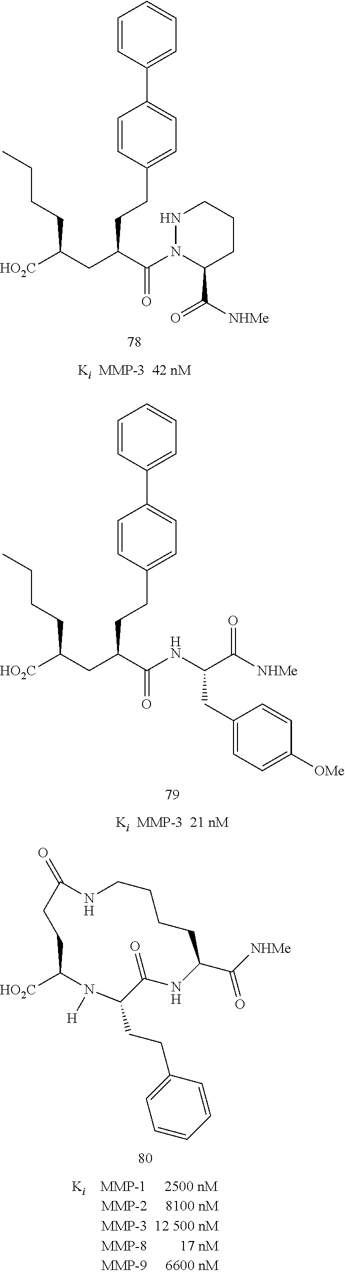

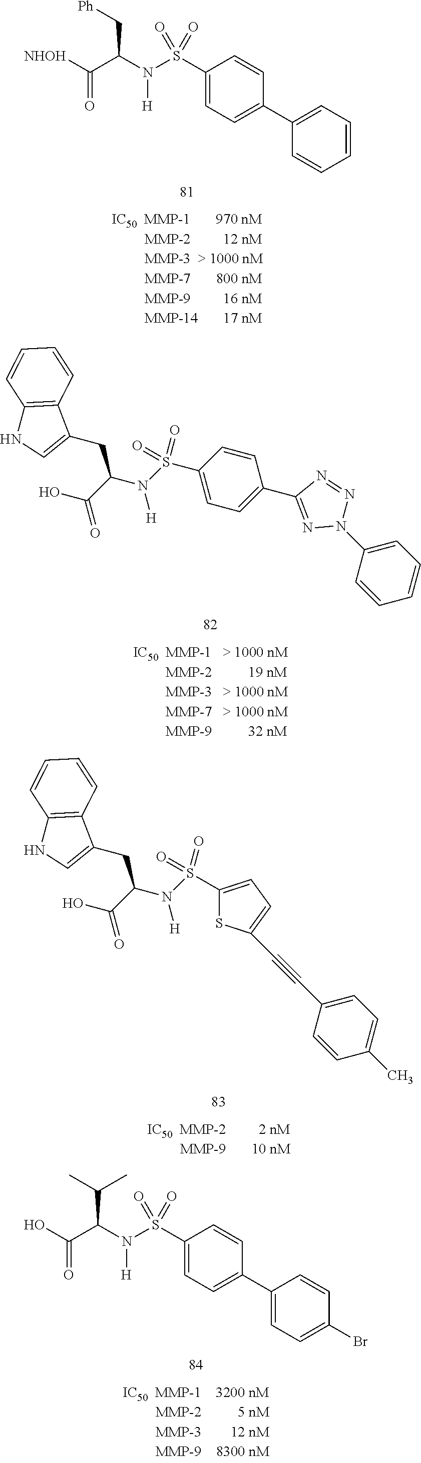

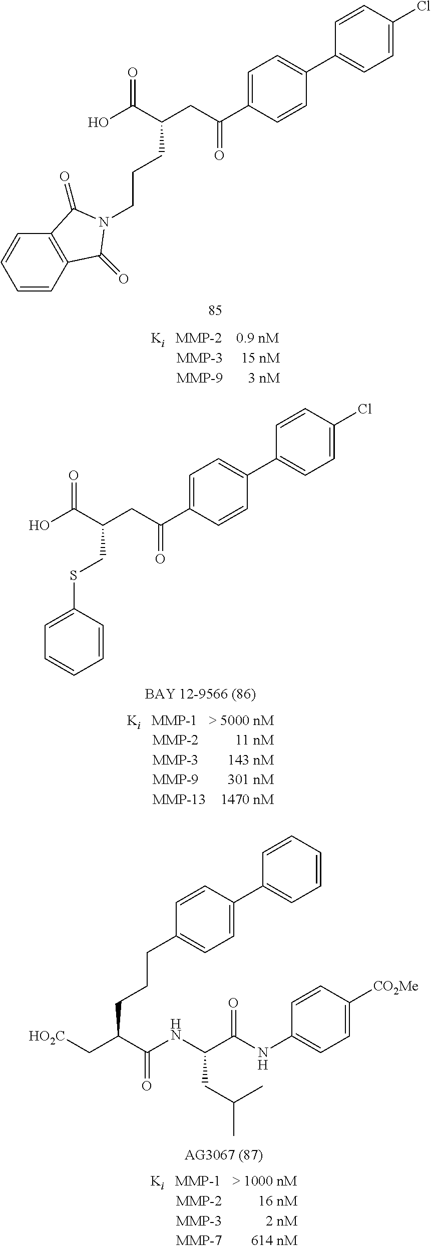

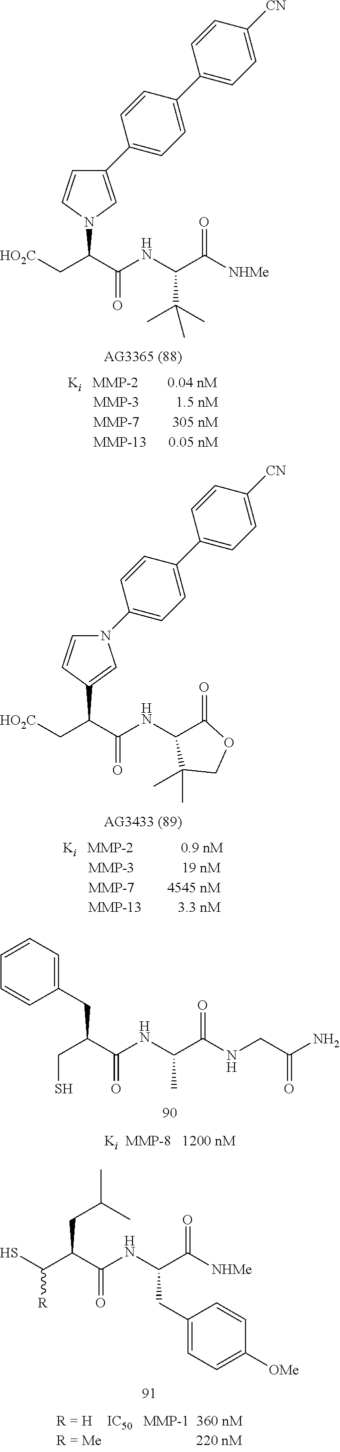

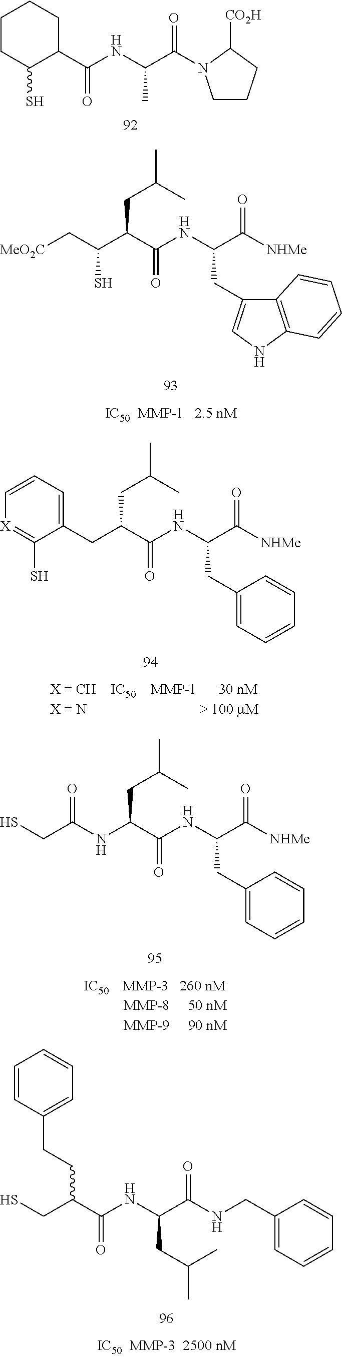

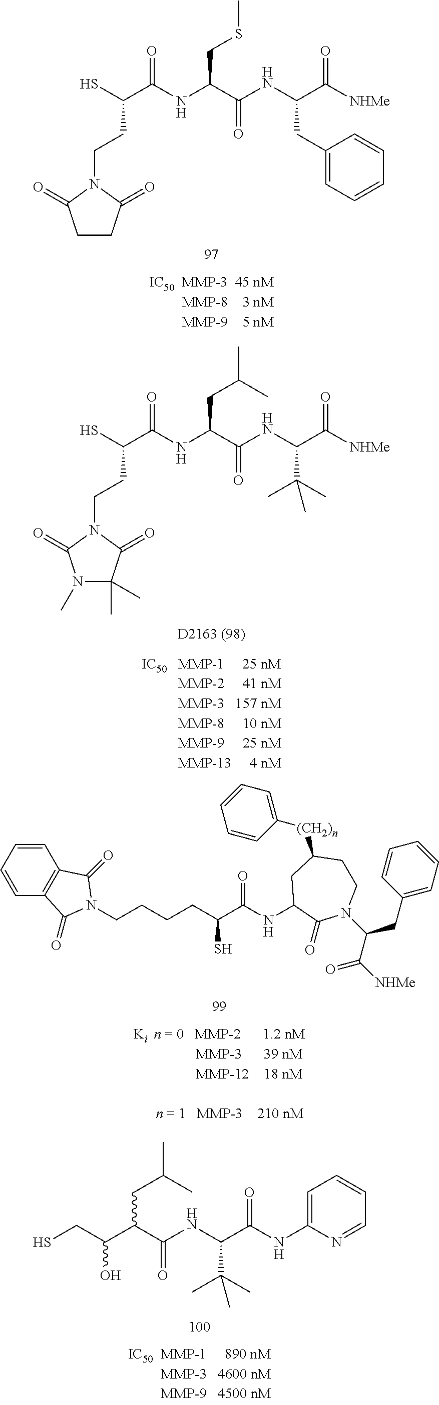

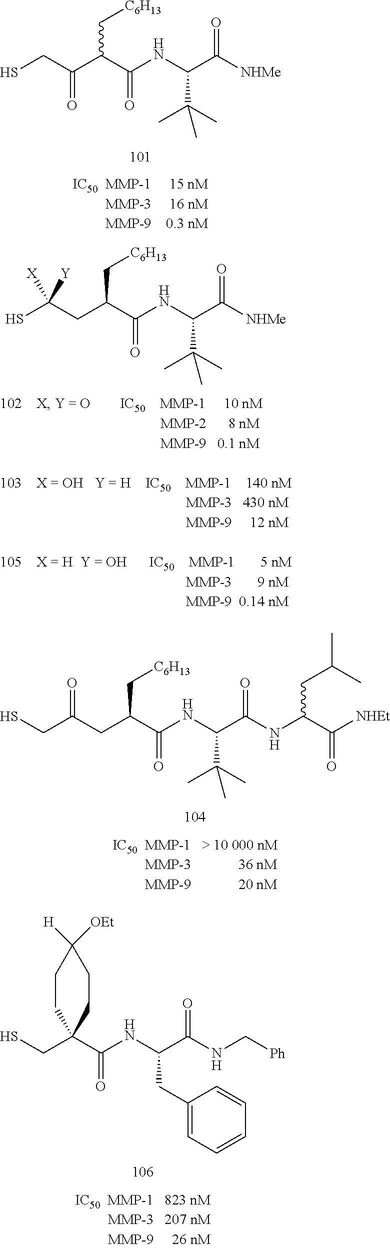

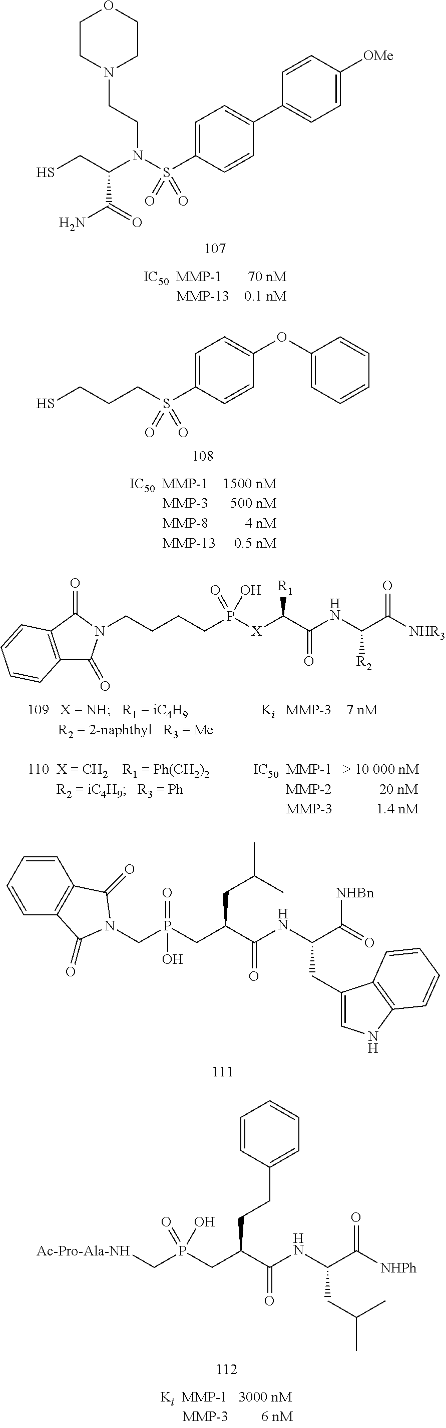

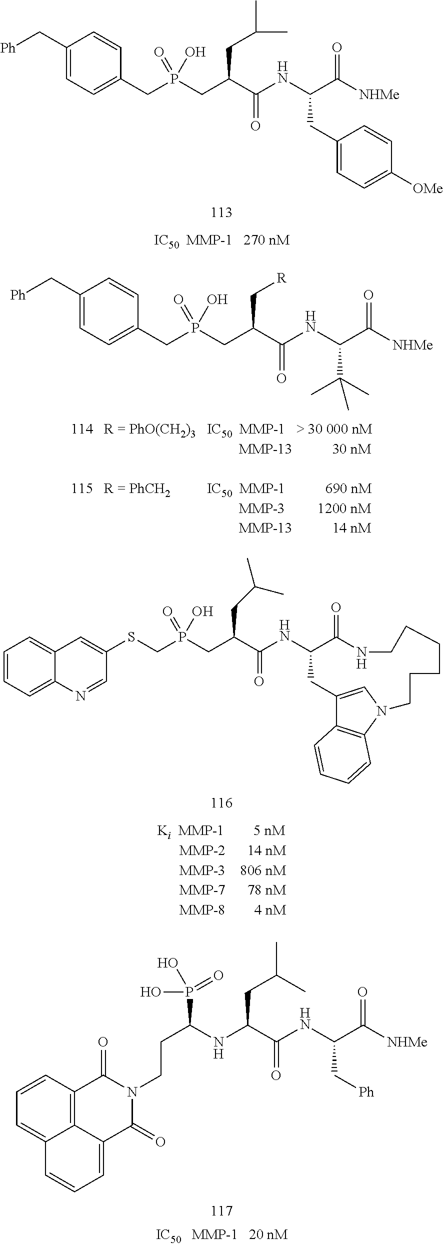

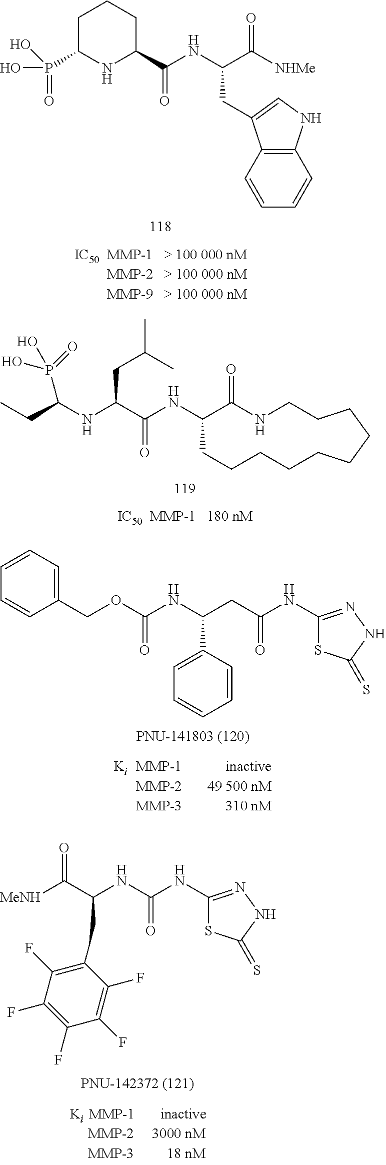

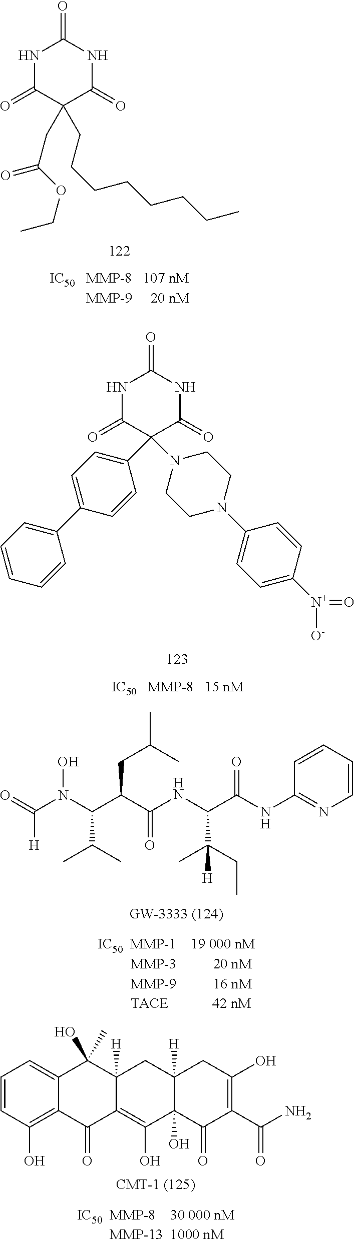

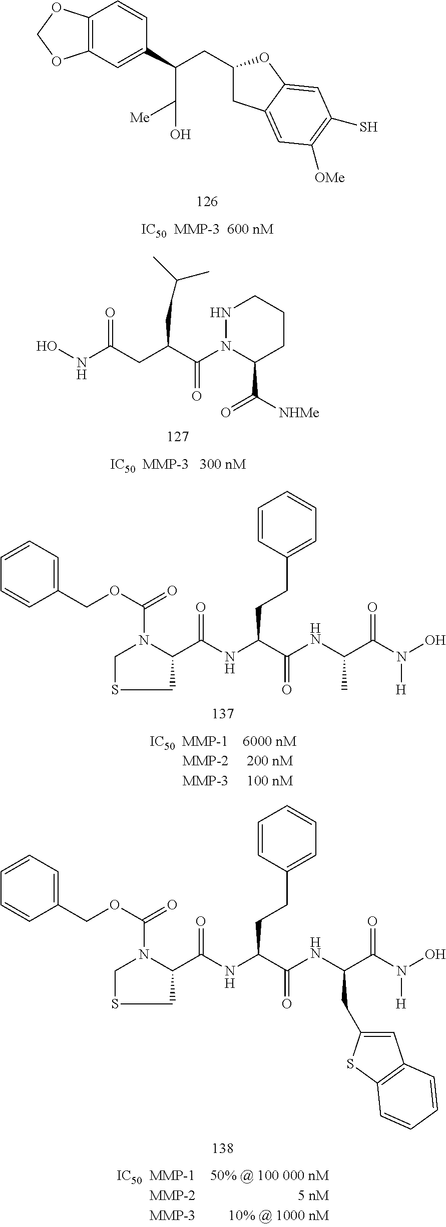

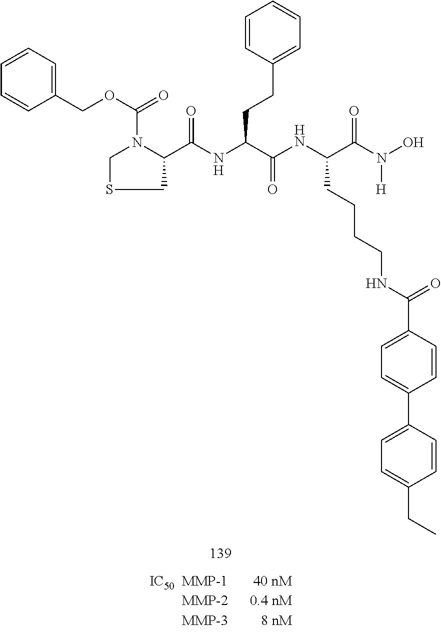

In one embodiment, the linker is a covalent bond. In certain embodiments, the linker is characterized by a first covalent bond or a chemical functional group that connects the chemical backbone moiety to a first end of the linker and a second covalent bond or chemical functional group that connects the ZBG to the second end of the linker. The first and second functionality, may or may not be independently present.

The linker, L can include linear or acyclic portions, cyclic portions, aromatic rings or combinations thereof. In certain embodiments, the linker can have from 1 to 50 main chain atoms other than hydrogen atoms, selected from C, N, O, S, P and Si. In certain embodiments the linker contains up to 20 main chain atoms other than hydrogen, up to 10, up to 5 or up to 2 main chain atoms other than hydrogen. In certain embodiments the linker is acyclic.

In one embodiment, a linker has functional groups that are used to interact with and form covalent bonds with the ZBG and the chemical backbone. Examples of functional groups include —NH₂, —NHNH₂, —ONH₂, —NHC═(O)NHNH₂, —OH, —CHO, halogen, —CO₂H, and —SH. Each of these functional groups can form a covalent linkage to a suitable functional group on the ZBG or the chemical backbone to obtain a compound of formula I. For example, amino, hydroxy and hydrazino groups can each form a covalent bond with a reactive carboxyl group (e.g., a carboxylic acid chloride or activated ester such as an N-hydroxysuccinimide ester (NHS)). Other suitable bond forming groups are well-known in the art.

In certain embodiments, R is

[[R¹]q-[O]p-[(R¹-[O]r-[(R²]o-[C(O)]s-[N(R^{a})]-[C(O)]t-[(R²]w-]

wherein q, p, r, o, s, t and w are each independently 0 or 1 and R^{a} is H, (C₁-C₄)alkyl, aryl or aralkyl; R¹ is aryl and R² is alkyl.

In one embodiment, R^{a} is phenyl, or benzyl

In certain embodiments R¹ is phenyl or 1,4-phenylene. In one embodiment, p, r, o, and w are 0 and s is 1. In one embodiment, t is 1. In one embodiment, t is 1 and w or o is 1. In one embodiment, p is 0, s is 0, t is 1 and w is 0. In one embodiment, q, p, r, and t or s are 0. In one embodiment, q is 1, p is 1, o is 0, s is 0, t is 1 and w is 0. In one embodiment, q is 0 or 1, p is 0 or 1, r is 0, o is 1, s is 0, t is 0 and w is 1. In one embodiment, q is 0 or 1, p is 0 or 1, r is 0, o is 0 or 1, s is 0 or 1, t is 0 or 1 and w is 1.

In one embodiment, R^{a} is H or —CH₃—.

In one embodiment, R is biphenylcarbamyl, biphenylcarbamylalkyl, biphenylalkylcarbamyl, biphenylalkylcarbamylalkyl, phenoxyphenylcarbamyl, arylalkylaminoalkyl, biphenylalkylaminoalkyl, arylcarbonylaminoalkyl, arylalkylcarbonylaminoalkyl, biphenyloxyalkylcarbonylaminoalkyl, or phenoxyphenylcarbamylalkyl, wherein, the phenyl or aryl group(s) may be optionally substituted, or a pharmaceutically acceptable salt thereof.

In one embodiment, R is

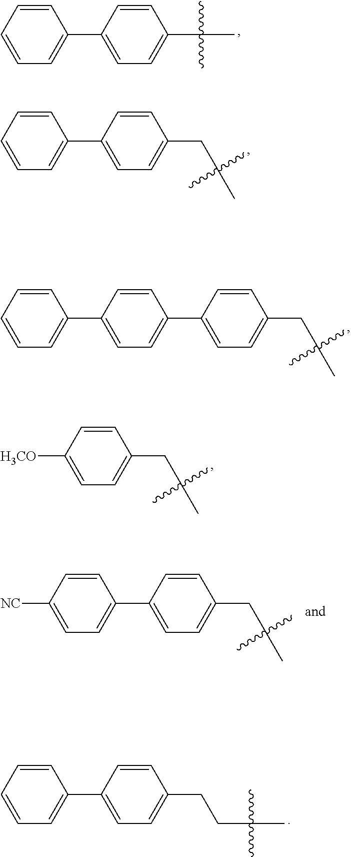

In certain embodiments, the R group is unsubstituted or substituted with one or more substituents, in one embodiment one to five substituents, in another embodiment one, two or three substituents, each independently selected from Q¹;

where Q¹ is hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano, isothiocyano, and each Q¹ is independently unsubstituted or substituted with one or more substituents, in one embodiment one, two or three substituents, each independently selected from Q²;

each Q² is independently hydrogen, halo, pseudohalo, hydroxy, oxo, thia, nitrile, nitro, formyl, mercapto, hydroxycarbonyl, alkyl, haloalkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, alkylcarbonyl, aminocarbonyl, alkoxy, aryloxy, heteroaryloxy, heterocyclyloxy, cycloalkoxy, alkenyloxy, alkynyloxy, aralkoxy, amino, aminoalkyl, alkylamino, arylamino, alkylthio, arylthio, thiocyano and isothiocyano.

In certain embodiments, the ZBGs provided herein are unsubstituted or substituted with one or more substituents, in one embodiment one to five substituents, in another embodiment one, two or three substituents, each independently selected from Q′; and each Q¹ is independently unsubstituted or substituted with one or more substituents, in one embodiment one, two or three substituents, each independently selected from Q², wherein Q¹ and Q² are as described above.

### C. Preparation of the Compounds

The compounds provided herein can be prepared by reactions known to one skilled in the art of chemical synthesis, for example, see, Puerta, et. al. J. Am. Chem. Soc. 2004, 126, 8388-8389 and Puerta, et. al., J. Am. Chem. Soc. 2005, 127, 14148-14149.

### D. Formulation of Pharmaceutical Compositions

The pharmaceutical compositions provided herein contain therapeutically effective amounts of one or more of compounds provided herein that are useful in the prevention, treatment, or amelioration of one or more of the symptoms of metalloprotein-mediated, including, but not limited to, matrix metalloproteinases (MMPs), histone deacetylases (HDACs) and anthrax lethal factor (LF) diseases.

The compositions contain one or more compounds provided herein. The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of metalloprotein-mediated, including, but not limited to, matrix metalloproteinases (MMPs), histone deacetylases (HDACs) and anthrax lethal factor (LF) diseases.

In certain embodiments, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of metalloprotein-mediated, including, but not limited to, matrix metalloproteinases (MMPs), histone deacetylases (HDACs) and anthrax lethal factor (LF) diseases.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing metalloprotein-mediated, including, but not limited to, matrix metalloproteinases (MMPs), histone deacetylases (HDACs) and anthrax lethal factor (LF) diseases. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets can be formulated. The compositions are in liquid, semi-liquid or solid form and are formulated in a manner suitable for each route of administration. Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. The pharmaceutically therapeutically active compounds and derivatives thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1 85% or about 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as metalloprotein-mediated, including, but not limited to, matrix metalloproteinases (MMPs), histone deacetylases (HDACs) and anthrax lethal factor (LF) diseases. It is to be understood that such combination therapy constitutes a further aspect of the compositions and methods of treatment provided herein.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate. Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

1. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide.

Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric Membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect. Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 gm.

Tablets and capsules for rectal administration are manufactured using the same pharmaceutically acceptable substance and by the same methods as for formulations for oral administration.

6. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

7. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

8. Articles of Manufacture

The compounds or pharmaceutically acceptable derivatives can be packaged as articles of manufacture containing packaging material, a compound or pharmaceutically acceptable derivative thereof provided herein, which is used for treatment, prevention or amelioration of one or more symptoms associated with metalloprotein-mediated, including, but not limited to, matrix metalloproteinases (MMPs), histone deacetylases (HDACs) and anthrax lethal factor (LF) activity, and a label that indicates that the compound or pharmaceutically acceptable derivative thereof is used for treatment, prevention or amelioration of one or more symptoms of metalloprotein-mediated, including, but not limited to, matrix metalloproteinases (MMPs), histone deacetylases (HDACs) and anthrax lethal factor (LF)-mediated diseases.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated.

### E. Evaluation of the Activity

The ability of compounds provided herein to inhibit the activity of matrix metalloproteinases (MMPs), histone deacetylases (HDACs) and anthrax lethal factor (LF) can be measured by methods known to one of skill in the art. In one embodiment, their efficacy against MMP-3 is evaluated using a fluorescent substrate assay as described in Knight et al., FEBS Lett., 1992, 296, 263-266. Examples 2 and 3 provide IC₅₀ values (in μM) for exemplary ZBGs against MMP-3 and anthrax LF measured using fluorescence-based assays.

### F. Methods of Use of the Compounds and Compositions

Methods of use of the compounds and compositions are also provided. The methods involve both in vitro and in vivo uses of the compounds and compositions.

In certain embodiments, provided herein are methods for inhibiting an action of metalloprotein, including, but not limited to matrix metalloproteinases, (MMPs), histone deacetylases (HDAC), and anthrax lethal factor (LF), by administering compounds and compositions provided herein. In one embodiment, the methods involve contacting the metalloprotein with a compound provided herein.

In other embodiments, provided herein are methods for treatment, prevention, or amelioration of one or more symptoms of diseases or conditions including, but not limited to conditions associated with metalloproteinases, including, but not limited to matrix metalloproteinases, (MMPs), histone deacetylases (HDAC), and anthrax lethal factor (LF) activity. The diseases include, but not limited to, proliferative diseases, autoimmune diseases, infectious diseases and inflammatory diseases. For example, diseases include e, but are not limited to, rheumatoid arthritis, lupus, multiple sclerosis, psoriasis, diabetic retinopathies, other ocular disorders, including recurrence of pterygii, scarring excimer laser surgery and glaucoma filtering surgery, various disorders of the anterior eye, cardiovascular disorders, restenosis, chronic inflammatory diseases, wounds, circulatory disorders, crest syndromes, bacterial infections, viral diseases, including AIDS, dermatological disorders, and cancer, including solid neoplasms and vascular tumors, including, but are not limited to, lung, colon, esophageal, breast, ovarian and prostate cancers.

In one embodiment, diseases associated with histone deacetylases, include, but are not limited to, cancer; psoriasis; fibroproliferative disorders; smooth muscle cell proliferation disorders; inflammatory diseases and conditions treatable by immune modulation; neurodegenerative disorders; diseases involving angiogenesis; fungal and parasitic infections; haematopoietic disorders; liver fibrosis; arteriosclerosis; restenosis; rheumatoid arthritis; autoimmune diabetes; lupus; allergies; Huntington's disease; retinal diseases, such as diabetic retinopathy, age-related macular degeneration, interstitial keratitis or rubeotic glaucoma; protozoal infections; anaemia; sickle cell anaemia; thalassemia and protozoal infections, such as malaria, toxoplasmosis or coccidiosis.

In certain embodiments, the compounds and compositions provided herein can be administered for therapeutic or prophylactic purposes together with another pharmacological agent known in the general art to be of value in treating one or more of the diseases or medical conditions referred to hereinabove, such as diseases or disorders associated with undesired and/or uncontrolled angiogenesis or neovascularization. It is to be understood that such combination therapy constitutes a further aspect of the compositions and methods of treatment provided herein.

The following examples are included for illustrative purposes only and are not intended to limit the scope of the subject matter claimed herein.

### EXAMPLES

Unless otherwise noted, starting materials were obtained from commercial suppliers and used without further purification. [(TpPh,Me)ZnOH] was synthesized as previously described (Puerta D. T.; Cohen S. M. Inorg. Chem. 2002, 41, 5075). Elemental analysis was performed at the NuMega Resonance Labs (San Diego, Calif.).

1H/13CNMR spectra were recorded on a Varian FT-NMR spectrometer running at 400 MHz at the Department of Chemistry and Biochemistry, University of California, San Diego. Infrared spectrum was collected on a Nicolet AVATAR 380 FT-IR instrument at the Department of Chemistry and Biochemistry, University of California, San Diego.

### Example 1

### Preparation of [(Tp^{Ph,Me})Zn(1)]

The probable mode of binding of zinc binding groups 1, 4, and 5 to the active site Zn²⁺ ion in MMPs, modeling studies were performed, see, Jacobsen, et al., Inorg. Chem. 2004, 43, 3038-3047; He, et al., Inorg. Chem. 2005, 44, 7431-7442 and He, et al.; Arif, A. M Inorg. Chem. 2004, 43, 2392-2401. A model complex of ZBG picolinic acid, 1, with [(Tp^{Ph,Me})Zn] (Tp^{Ph,Me}=hydrotris(3,5-phenylmethylpyrazolyl)borate) was prepared as follows:

In a 50 mL round-bottom flask, [(Tp^{Ph,Me})ZnOH] (100 mg, 0.176 mmol) was added to 19 mL of CH₂Cl₂. To this solution was added 1.0 equiv of ZBG 1 (21.6 mg, 0.176 mmol) dissolved in 10 mL of methanol. The mixture was stirred at room temperature overnight under a nitrogen atmosphere. After stirring, the solution was evaporated to dryness on a rotary evaporator to give a white solid. The solid was dissolved in a minimum amount of benzene (˜3 mL), filtered to remove any insoluble material, and the filtrate was recrystallized by diffusion of the solution with pentane.

Yield: 88%. ¹HNMR (CDCl₃, 400 MHz, 25° C.): δ 2.59 (s, 9H, pyrazole-CH₃), 6.23 (s, 3H, pyrazole-H), 7.34 (m, 1H, phenyl-H), 7.68 (d, 6H, phenyl-H), 7.85 (m, 1H, phenyl-H), 7.96 (m, 1H, phenyl-H), 8.32 (m, 1H, phenyl-H). 13CNMR (CDCl₃, 100 MHz, 25° C.): δ 13.3, 104.9, 105.6, 122.9, 125.9, 127.6, 128.3, 128.6, 128.9, 129.1, 132.3, 145.6, 153.5. IR (film from CH₂Cl₂): v 694, 774, 1059, 1167, 1344, 1671, 2539

X-Ray Crystallographic Analysis.

Colorless blocks suitable for X-ray diffraction were grown from a solution of the complex in benzene diffused with pentane. A crystal was mounted on a quartz capillary by using Paratone oil and cooled in a nitrogen stream on the diffractometer. Data was collected on a Bruker AXS diffractometer equipped with area detectors. Peak integrations were performed with the Siemens SAINT software package. Absorption corrections were applied using the program SADABS. Space group determination was performed by the program XPREP. The structure was solved by direct methods and refined with the SHELXTL software package. All hydrogen atoms were fixed at calculated positions with isotropic thermal parameters and all nonhydrogen atoms were refined anisotropically. The hydrogen atom on the boron atom was found in the difference map and the position was refined. The compound co-crystallized with a one equivalent of benzene per complex.

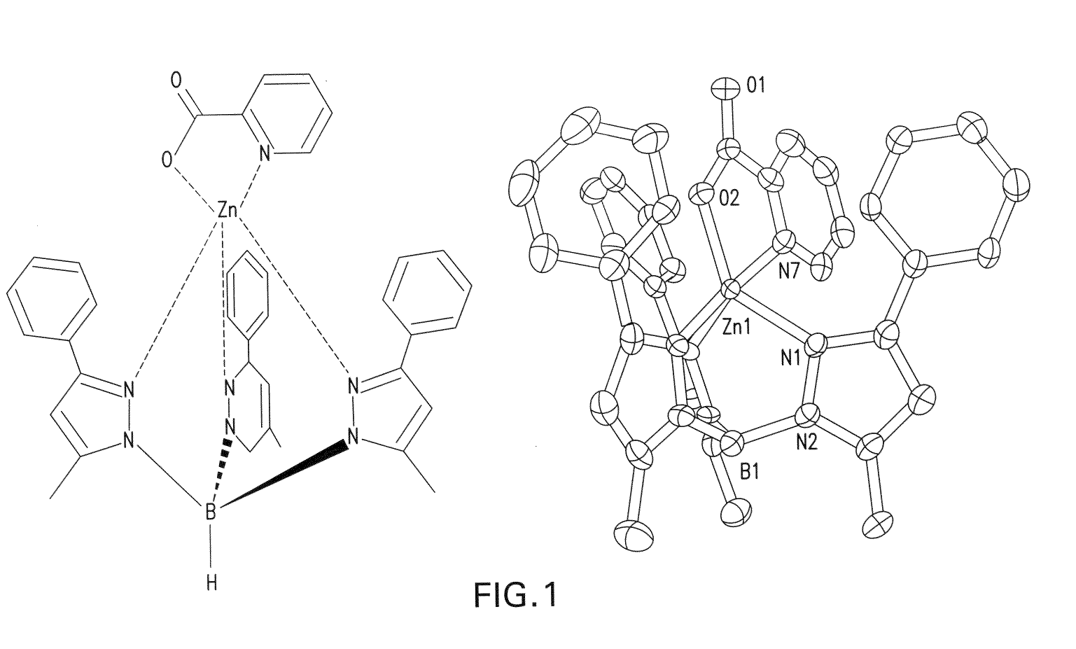

The complex (FIG. 1) shows that picolinic acid binds to the active site model in a bidentate fashion through the pyridyl nitrogen atom and the deprotonated carboxylate oxygen atom (Table 1). To elucidate the mode of binding for 4 and 5, several attempts were made to prepare [(TpPh,Me)Zn(4)] and [(TpPh,Me)Zn(5)] using a variety of reaction conditions. In all cases the reactions produced [Zn(ZBG)₂]²⁺, indicating that these ligands are sufficiently strong chelators for Zn²⁺ that they strip the metal ion from [(TpPh,Me)Zn(OH)].

TABLE 1
X-ray structure data for the complexes [(TpPh,Me)Zn(1)].

| | [(Tp)Zn(1)] |
|---|---|
| Empirical Formula | C₄₂H₃₈BN₇O₂Zn |
| Crystal System | Orthorhombic |
| Space Group | P2₁2₁2₁ |
| Unit Cell dimensions | a = 8.8038(5) Å b = |
| | 14.5333(7) Å c = |
| | 29.4585(15) Å |
| | α = β = γ = 90° |
| Volume, Z | 3769.2(3) Å₃, 4 |
| Crystal size | 0.25 × 0.20 × 0.10 mm₃ |
| Temperature (K) | 100(2) |
| Reflections collected | 23369 |
| Independent reflections | 8382 [R(int) = 0.0214] |
| Data/restraints/parameters | 8382/0/485 |
| Goodness-of-fit on F₂ | 1.043 |
| Final Rindices I > 2σ(I)ₐ | R1 = 0.0288 |
| | wR2 = 0.0726 |
| Rindices (all data)ₐ | R1 = 0.0310 |
| | wR2 = 0.0736 |

ₐΣΣ −= _{oco}FFFR/_{1, 2/14222 2}][/])([ΣΣ −= _{oco}wFFF w R

### Example 2

### Fluorescent MMP Assays

MMP-3 and MMP-1 activity of the ZBGs were measured utilizing a 96-well microplate fluorescent assay kit purchased from Biomol Research Laboratories, following the procedure provided with the kit. Experiments were performed using a Bio-Tek Flx 800 fluorescence plate reader and Nunic white 96-well plates. ZBGs 1, 4, and 5 were dissolved in DMSO and further diluted (500×) into the assay buffer (MMP-3: 50 mM MES, 10 mM CaCl₂, 0.05% Brij-35, pH 6.0; MMP-1: 50 mM HEPES, 10 mM CaCl₂, 0.05% Brij-35, pH 7.5). Inhibitors 2, 3, 6, and 7 were dissolved directly into assay buffer. MMP-1 and MMP-3 were incubated with varying concentrations of inhibitors for 1 h at 37° C., followed by addition of substrate to initiate the assay. The reactions were agitated by shaking for 1 sec after each fluorescence measurement. Upon cleavage of the fluorescent substrate Mca-Pro-Leu-Gly-Leu-Dpa-Ala-Arg-NH₂ (0.4 mM concentration in assay, Mca=(7-methoxycoumarin-4-yl)-acetyl, Dpa=N-3-(2,4-dinitrophenyl)-L-α-β-diaminopropionyl) at the Gly-Leu bond, Mca fluorescence (λex=335 nm, λem=405 nm) was measured at 60-second intervals for 20 minutes for MMP-3 and 60 minutes for MMP-1.

Table 2 provides IC₅₀ values (in μM) for ZBGs against MMP-3 as compared with a reference compound acetohydroxamic acid (AHA); AHA represents the most commonly used ligand in MMPi.

TABLE 2
IC₅₀ Values (μM) for ZBGs against MMP-3 and Anthrax LF
measured using Fluorescence-Based Assays

| ZBG | MMP-3 IC_{50 a} | LF IC_{50 a} | potency v. AHA_{b} |
|---|---|---|---|
| AHA | 25100 ((4000) | 11400 ((1000) | n/a |
| 1 | 181 ((10) | 3500 ((300) | 139-fold |
| 2 | 1350 ((160) | 1275 ((7) | 19-fold |
| 3 | 6100 ((200) | 2690 ((80) | 4-fold |
| 4 | 154 ((13) | 5700 ((700) | 163-fold |
| 5 | 136 ((9) | 370 ((40) | 185-fold |
| 6 | 180 ((30) | 930 ((30) | 139-fold |
| 7 | 1330 ((140) | 2920 ((80) | 19-fold |

ₐObtained from at least three independent experiments;
_{b}Based on IC₅₀ value from MMP-3 fluorescence assay.

Table 3 provides IC₅₀ values for ZBGs provided herein against MMP-1 measured by using a fluorescence-based assay. All values obtained from at least three independent experiments.

TABLE 3
IC₅₀ values for ZBGs provided herein against MMP-1

| | MMP-1 (μM) | |
|---|---|---|
| ZBG | IC₅₀ | Potency v. AHA |
| AHA | 41600 (±400) | n/a |
| 1 | 2500 (±150) | 17-fold |
| 2 | 5688 (±79) | 7-fold |
| 3 | 13480 (±710) | 3-fold |
| 4 | 430 (±10) | 98-fold |
| 5 | 63 (±4) | 660-fold |
| 6 | 510 (±60) | 82-fold |
| 7 | 3490 (±120) | 12-fold |

### Example 3

### Recombinant Anthrax Lethal Factor Assays

Activities of Bacillus anthracis recombinant anthrax lethal factor (Calbiochem) were measured following literature procedures with some modifications (R. T. Cummings et. al, Proc. Nat. Acad. Sci. USA, 2002, 99, 6603-6606). Experiments were performed using a Bio-Tek Flx 800 fluorescence plate reader and Nunc white 96-well plates. ZBGs 1, 4, and 5 were dissolved in DMSO and further diluted (500×) into the assay buffer (20 mM HEPES, 1.0 mM CaCl₂, 0.1 mg/ml BSA, 0.1% Tween-20, pH 7.0). ZBGs 2, 3, 6, and 7 were dissolved directly into assay buffer. LF (3 nM final concentration in assay) was incubated with varying concentrations of different inhibitors for 45 min at 25° C., followed by addition of substrate to initiate the assay. Reaction's were agitated by shaking for 1 sec after each fluorescence measurement. Upon cleavage of the fluorescent substrate, (Cou)-N-Nle-Lys-Lys-Lys-Lys-Val-Leu-Pro-Ile-Gln-Leu-Asn-Ala-Ala-Thr-Asp-Lys-(QSY-35)-Gly-Gly-NH₂ (0.75 μM in assay; Cou=7-hydroxy 4-methyl-3-acetylcoumarinyl; QSY-35=N-((4-((7-nitro-2,1,3-benzoxzdiazol-4-yl)amino)phenyl)acetyl), at the Pro-Ile bond the Cou fluorescence was measured at 60-second intervals for 20 min (λex=380 nm, λem=450 nm). Experiments were repeated in at least triplicate. IC₅₀ values were calculated as the inhibitor concentration at which the enzyme is at 50% control activity (no inhibitor present).

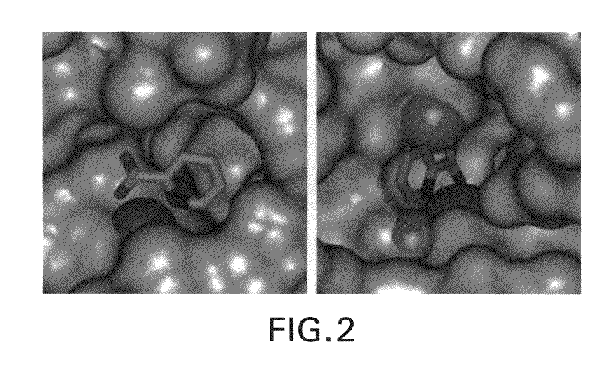

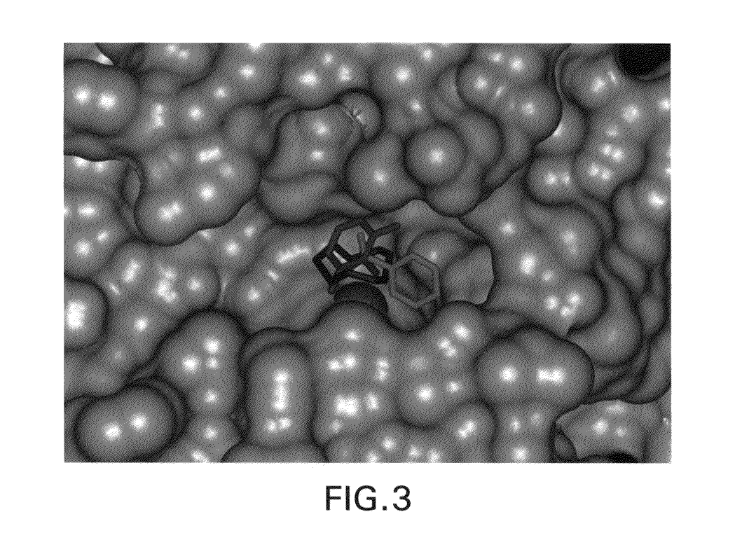

As shown in Table 2, ZBGs 1-7 demonstrate greater inhibition of anthrax lethal factor than AHA. These ZBGs show greater potency against MMP-3 than against LF. This difference may be due in part to the rather closed active site of LF, relative to those of the MMPs. This is illustrated by taking the structural parameters (the Zn²⁺ coordination environment and ZBG) from [(Tp^{Ph,Me})Zn(1)] and superimposing them into the crystallographically determined MMP-3 (1G4K) and LF (1PWQ) active sites. Steric interactions between the ZBG and the protein active site were assessed with the program InsightII (FIG. 3). As shown in FIG. 2, ZBG 1 clearly clashes with the LF protein surface, but has no such steric conflicts in the MMP-3 active site.

### Example 4

### Lipoxygenase Assay

The potential selectivity of the ZBGs provided herein was examined by studying the inhibition of the non-heme iron enzyme soybean lipoxygenase (sbLO) by these compounds.

Linoleic acid and type 1-B soybean lipoxygenase as a lyophilized power at >100,000 U/mg were purchased from Sigma-Aldrich. Taking into account the 60% stabilizers in the lyophilized powder a stock solution of 10,000 U/mL protein in 0.1 M borate buffer (pH 9) was prepared. Soybean lipoxygenase (final concentration 250 U/1.5 mL reaction volume) was preincubated with chelator (diluted from 1 M DMSO stock; final concentration 100 μM) or respective dilutent for 3 hours. After preincubation the reaction was initiated by the addition of linoleic acid (diluted from 3.21 M ethanol stock solution; final concentration 667 μM). The rate of reaction was monitored by increase in absorbance at 234 nm over 20 min. Percent activity was determined by dividing the slope of samples with inhibitors by the slope of control samples.

The reference compounds AHA and maltol (300 μM) were found to inhibit product formation (↑ 99% and 70%, respectively) relative to control (no inhibitor). In contrast, compounds 1 and 7 had essentially no effect on lipoxygenase activity (2.3% and <1% inhibition, respectively). These results suggest that nitrogen-based ZBGs will show improved specificity for Zn²⁺ metalloproteinase over enzymes dependent on other metal ions, such as iron.

Since modifications will be apparent to those of skill in the art, it is intended that the claimed subject matter be limited only by the scope of the appended claims.

1. A metalloprotein inhibitor compound of Formula I:
(R)ₙ-L-ZBG,
wherein R is a chemical backbone for generating noncovalent interactions within the metalloprotein active site pocket;
L is a linker that connects the chemical backbone with the zinc binding group;
n is 1-4; and
ZBG is a zinc binding group comprising a nitrogen or mixed nitrogen/oxygen donor-atom chelator.

2. The compound of claim 1, wherein the compound has Formula II:
R-L-ZBG.

3. The compound of claim 1, wherein the ZBG comprises a nitrogen donor atom.

4. The compound of claim 1, wherein the ZBG comprises a nitrogen and a oxygen donor-atom.

5. The compound of claim 1, wherein the ZBG is

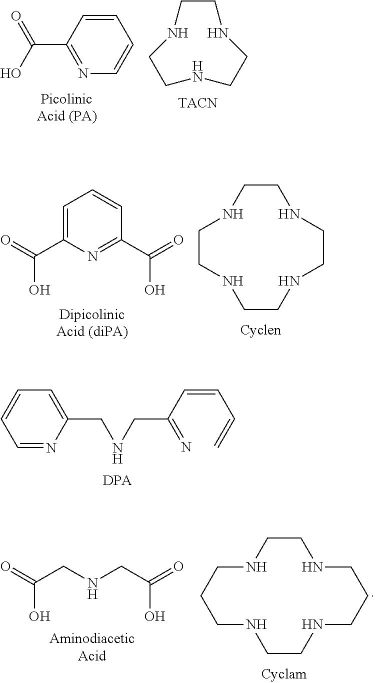

6. The compound of claim 1, wherein R is a peptide, peptidomimetic or other organic moiety suitable for a noncovalent interaction in the metalloprotein active site pocket.

7. The compound of claim 1, wherein R is
[(R¹)q-[O]p-(R¹)—[O]r-(R²)o-[C(O)]s-[N(R^{a})]-[C(O)]t-(R²)w]
wherein q, p, r, o, s, t and w are each independently 0 or 1 and R¹ is aryl, R² is alkyl and R^{a} is H, alkyl, or aryl.

8. The compound of claim 7, wherein R¹ is phenyl or 1,4-phenylene.

9. The compound of claim 7, wherein p, r, o, and w are 0 and s is 1.

10. The compound of claim 7, wherein R is biphenylcarbamyl, biphenylcarbamylalkyl, biphenylalkylcarbamyl, biphenylalkylcarbamylalkyl, phenoxyalkylcarbamyl, phenylalkylaminoalkyl, biphenylalkylaminoalkyl, phenylcarbonylaminoalkyl, phenylalkylcarbonylaminoalkyl, biphenyloxyalkylcarbonylaminoalkyl, or phenoxyphenylcarbamylalkyl.

11. The compound of claim 7, wherein R is

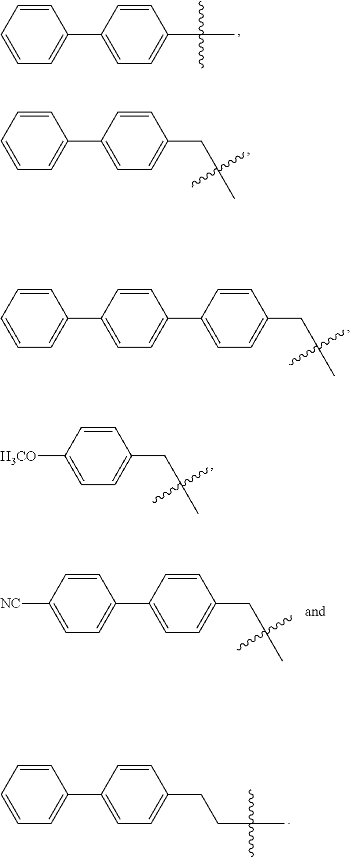

12. A pharmaceutical composition comprising a compound of claim 1 and a pharmaceutically acceptable carrier.

13. A method for inhibiting an action of a metalloproteinase, comprising contacting the metalloprotein with a compound of claim 1.

14. The method of claim 13, wherein the metalloprotein is a matrix metalloproteinase, histone deacetylases or anthrax lethal factor.

15. A method for treatment, prevention, or amelioration of one or more symptoms of a disease associated with a metalloprotein, comprising administering a compound of claim 1.

16. The method of claim 15, wherein the disease is selected from a proliferative disease, autoimmune disease, infectious disease and inflammatory disease.

17. The method of claim 16, wherein the disease is cancer; psoriasis; fibroproliferative disorders; smooth muscle cell proliferation disorders; inflammatory diseases and conditions treatable by immune modulation; neurodegenerative disorders; diseases involving angiogenesis; fungal and parasitic infections; haematopoietic disorders; liver fibrosis; arteriosclerosis; restenosis; rheumatoid arthritis; autoimmune diabetes; lupus; allergies; Huntington's disease; retinal disease; protozoal infection; anaemia; thalassemia and protozoal infection.

18. An article of manufacture, comprising packaging material and a compound of claim 1 contained within the packaging material, wherein the compound is for treating, preventing or ameliorating a condition associated with metalloprotein and the packaging material includes a label that indicates that the compound is used for treating, preventing or ameliorating a condition associated with metalloprotein.
